(19) United States
(12) Patent Application Publication
(10) Pub. No.: US 2005/0064450 A1
(43) Pub. Date: Mar. 24, 2005

(54) Methods of making repetitive sequences removed probes and uses thereof

(76) Inventors: Joe N. Lucas, San Ramon, CA (US); Zhong Chen, Sandy, UT (US)

Correspondence Address: LAW OFFICES OF KHALILIAN SIRA, LLC, 330 ALDERWOOD DRIVE, GAITHERSBURG, MD, 20878, US

(21) Appl. No.: 10/798,949

(22) Filed: Mar. 12, 2004

Related U.S. Application Data
(60) Provisional application No. 60/453,962, filed on Mar. 13, 2003.

(51) Int. Cl. C12Q 1/68; C12P 19/34

(52) U.S. Cl. 435/6; 435/91.2

(57) ABSTRACT

The invention discloses novel methods and compositions for the detection of a target nucleic acid molecule in a sample. In particular, the invention provides a method of producing a probe having removed repetitive sequences comprising: (a) providing a source nucleic acid molecule containing repetitive sequences; (b) providing a driver nucleic acid molecule attached to a label and containing repetitive sequences that hybridize with the repetitive sequences of the source nucleic acid molecule; (c) hybridizing the source nucleic acid molecule and the driver nucleic acid molecule in the presence of a molecule that binds the label of step (b) wherein the repetitive sequences of source nucleic acid molecule hybridize with the repetitive sequences of the driver nucleic acid molecule to form a product; (d) subtracting the hybridized repetitive sequences of the product of step (c) by extraction with a protein dissolving solution to remove the hybridized repetitive sequences from the product; and (e) recovering the probe having repetitive sequences removed therefrom.

### CROSS REFERENCE TO RELATED APPLICATION

This application claims the benefit of U.S. provisional patent application Ser. No. 60/453,962, filed Mar. 13, 2003, content of which is incorporated herein by reference in its entirety.

### I. FIELD OF THE INVENTION

The present invention relates to methods and compositions for generating unique nucleic acid probes for detection of target molecules in a sample. More specifically, the present invention relates to a method for production of probes having repetitive sequences removed therefrom.

### II. BACKGROUND OF THE INVENTION

It has been known for decades that chromosome rearrangements exist in most, if not all, human cancers (Miteiman et al., Cytogenetic Cell Genet, 58:653-79 (1991)) and certain human hereditary diseases (Frezal et al., Cytogenetic Cell Genet, 58:986-1052 (1991)). Distinct chromosomal abnormalities in cancers lead to the activation of proto-oncogene products, creation of cancer-specific fusion proteins, or inactivation of tumor suppressor genes.

Since chromosome-banding techniques were developed, cytogenetic analysis of nonrandom chromosome abnormalities in malignant cells has become an integral part of the diagnostic and prognostic work of many human cancers (Sandberg, The Chromosomes In Human Cancer And Leukemia, Elsevier; New York, pp. 10 (1990)). Additionally, cytogenetic studies followed by molecular analysis of recurring chromosomal rearrangements have greatly facilitated the identification of genes related to the pathogenesis of both hereditary disease and cancer. For example, the tumor suppressor gene, Rb-1, was identified based on the observation of deletion of chromosome 13q14 in retinoblastoma (Yunis and Ramsay, Am. J. Dis. Child. 132:161-163 (1978)) and the proto-oncogene, c-myc, was shown to be involved in the chromosome translocation t(8 and 14) in human Burkitt's lymphomas (Zech, et al., Int. J. Cancer 17:47-56 (1976)).

However, not all cytogenetically visible chromosome rearrangements (i.e., complex chromosome rearrangements, small ring chromosomes, and unidentifiable de novo unbalanced translocations) can be determined by conventional cytogenetic banding analysis. This technique limitation that prevents complete karyotypic analysis in many human cancers, particularly solid tumors has been countered by the development of fluorescence in situ hybridization (FISH) techniques (Pinkel, et al., Proc. Natl. Acad. Sci. USA 85: 9138-42 (1988)). After more than a decade of effort, a variety of fluorescent DNA probes, such as painting probes including human whole chromosome painting probes (WCPs) (Guan, et al., Genomics, 22(1):101-107 (1994)), chromosome arm panting probes (CAPs) (Guan et al., Nature Genet 12: 10-11 (1996)), and chromosome band-specific painting probes (Guan et al., Clinic Cancer Res., 1: 11-18 (1995)), have been developed and widely applied in both research and clinical diagnostics.

A major problem with currently available fluorescent painting probes and other genomic DNA probes, such as yeast artificial chromosome (YAC) and bacterial artificial chromosome (BAC) (Thompson & Thompson Genetics in Medicine, 6th ed. Thompson M W, Mcinnes R R, Willard H F, eds. W. B. Saunders, 2001) containing human genomic DNA fragments, is the background signals caused by the cross-hybridization of the repetitive sequences existing in the probes. Human genomic DNA contains many different types of repetitive sequences. Some of these sequences such as the short highly repetitive sequences Alu and the long repetitive sequences Li, appear in genomic DNA approximately every few kilo-bases. One solution has been to block these repetitive sequences during hybridization. Conventional blocking methods have been used in which commercially available human Cot-1 DNA containing several different repetitive sequences is applied to pre-hybridization solution containing a probe with repetitive sequences.

Conventional blocking methods, however, suffer from many drawbacks. First, the pre-hybridization process tends to decrease the fluorescent signals due to self-hybridization of the unique sequences in the probe before hybridization to the target sequences. Second, the process is cumbersome and time consuming. Substantial time and effort have been used to determine the optimal ratio of Cot-1 to each DNA probe during commercial preparation for hybridization. The problem was exacerbated for preparing probes for multi-color FISH and Fast-FISH probes, since these methods require higher quality probes with less noise compared to conventional probes. Finally, the human Cot-1 DNA is cost-prohibitive.

A more specific and efficient method is needed to remove repetitive sequences from nucleic acid probes while preserving the unique sequences. The invention as disclosed and described herein, overcomes the prior art problems with the generation of probes having removed repetitive sequences therefrom with increased accuracy and specificity and efficiency.

### III. SUMMARY OF THE INVENTION

The invention, as disclosed and described herein, provides repetitive sequences removed probes (RSRPs), method of making and method of using these probes.

In one aspect, the method of making RSRPs comprises (a) providing a source nucleic acid molecule containing repetitive sequences; (b) providing a driver nucleic acid molecule attached to a label and containing repetitive sequences that hybridizes with the repetitive sequences of the source nucleic acid molecule, (c) hybridizing the source nucleic acid molecule and the driver nucleic acid molecule in the presence of a molecule that binds the label of step (b) wherein the repetitive sequences of source nucleic acid molecule hybridize with the repetitive sequences of the driver nucleic acid molecule to form a product; (d) subtracting the hybridized repetitive sequences by extraction with a protein dissolving solution to remove the hybridized repetitive sequences from the product; and (e) recovering the probe having repetitive sequences removed therefrom.

In another embodiment, the recovered probes having reduced or substantially removed repetitive sequences are processed one or more times through steps (a) to (e). Removed repetitive sequences or substantially removed repetitive sequences refer to at least about 60%, preferably about 75%, more preferably about 90%, 91%, 92%, 93%, 94%, 95%, 96%, 97%, 98%, 99%, and most preferably about 100% removed repetitive sequences.

In a preferred embodiment, the driver DNA has biotin-labeled repetitive sequences. After the reaction has been completed, the hybridized repetitive sequences are removed in step (d) using the novel two-step procedure of the invention: (i) incubating the product of step (c) with, for example, avidin and subtracting the hybridized repetitive sequences with phenol and (ii) incubating the product of step (i) with avidin-labeled magnetic beads in the binding buffer of the invention, and thereby removing the hybridized remaining repetitive sequences by concentrating the beads under a magnetic force. In one embodiment, step (ii) is performed prior to step (i). The addition of a salt of a weak acid, i.e., sodium acetate, improves the separation. The final repetitive sequences removed probe is recovered as a precipitate by amplification.

In one embodiment, the removed repetitive sequences are recovered by PCR using unique-sequence primers. The unique sequence primers comprise DL1, DL2, nucleic acid molecules that are substantially homologous to D1 and D2, or nucleic acid molecules that hybridize under stringent conditions with D1 and/or or D2.

In yet another embodiment, the invention provides methods and compositions for detecting nucleic acid sequences in a variety of applications. For example, the methods and compositions of the invention are used in the detection of chromosomal abnormalities, detection of genetic diseases, detection of cancer, detection of bacterial or viral infections, determination of a genetic relationship, such as paternity or species identification, determination of potential donors of organs or tissues, among others.

In a preferred embodiment, the compositions and methods of the invention are used to detect benign, chronic or acute cancers. In this case, preferably the repetitive sequences removed probes of the invention are derived from a source DNA that comprises a gene probe for cancer, including, for example, leukemia, retinoblastoma, human Burkitt's lymphomas, ovarian cancer, uterine cancer, prostate cancer, breast cancer, among others.

In yet another aspect, the invention provides a nucleic acid molecule comprising DL1, represented by SEQ ID NO: 2; DL2 represented by SEQ ID No: 3, or a sequence that is substantially homologous to SEQ ID NO: 2, or SEQ ID NO:3.

In another aspect, the invention provides a diagnostic test kit for the detection of target nucleic acid molecules in a sample. In one embodiment, the diagnostic test kit detects chromosomal abnormalities in a patient's sample and comprises one or more repetitive sequences removed probes (RSRPs) that specifically detect chromosomal abnormalities and a detection agent comprising a detectable label.

It is still another aspect of the present invention to provide for the generation of removed repetitive sequences probe libraries that meet the demands of advanced FISH technologies, such as FAST FISH and MULTI-COLOR FISH, decrease the cost of manufacture of such probes, and simplify the protocols for using these probes in FISH.

### IV. BRIEF DESCRIPTION OF THE DRAWINGS

FIG. 1. Effective subtraction of avidin-biotin labeled complexes. Before subtraction, a smear of 200-1000 bp DNA was observed. The precipitated product after the 1st round of subtraction (lane 2), and after the second round of subtraction (lane 3) was compared to the sample before subtraction. As shown, the majority of biotin-labeled driver DNA was removed after the 1st subtraction (lane 2). Lane 1 is the molecular marker.

FIG. 2. Recovery of RSRPs from 15q by PCR using DL2 primer. When biotinylated microdissected DNA (lane 1) was used in the subtraction procedure, the added avidin bound to the DNA. The majority of DNA was removed after the 1st round of subtraction (lane 2) and some was removed after the 2nd round of subtraction (lane 3). When non-labeled microdissected DNA (lane 4) was used in the procedure, the added avidin did not bind to the DNA. The DNA was efficiently recovered after the 1st (lane 5), and after the 2nd rounds of subtraction (lane 6). These results demonstrate the feasibility of recovering the non-labeled microdissected DNA following multiple rounds of subtraction.

FIG. 3. Detection of repetitive sequences by Southern blot analysis. Panel A: The gel picture shows equal amounts of microdissected DNA loaded on gel from 9q, 12p, and 15q, lanes 1, 4, and 7, respectively before subtraction; lanes 2, 5, and 8, respectively after first subtraction; and lanes 3, 6, and 9, respectively after 2nd subtraction. Panel B: Hybridization with ³²P labeled Cot-1 DNA. Microdissected DNA of 9q, 12p, and 15q before subtraction are shown in lanes 1, 4, and 7, respectively; lanes 2, 5, and 8, respectively after first subtraction; and lanes 3, 6, and 9, respectively after 2nd subtraction. The hybridization results demonstrate the efficient removal of the repetitive elements. The majority of the repetitive sequences were removed after the first round of subtraction.

FIG. 4. Comparison between the sizes of the PCR amplified products after amplification with DL1 and DL2 primers. Lane 1: MW marker; Lane 2: DL1 amplified DNA from 9q; Lane 3, DL2 amplified DNA from 9q; Lane 4: DL1 amplified DNA from 12p; Lane 5, DL2 amplified DNA from 12p; Lane 6: DL1 amplified DNA from 15q; Lane 7: DL2 amplified DNA from 15q; Lane 8: DL1 amplified DNA from 12qter; Lane 9: DL2 amplified DNA from 12qter; Lane 10: DL1 amplified DNA from 18qter; Lane 11: DL2 amplified DNA from 18qter; Lane 12: DL1 amplified DNA from 5p; Lane 13: DL2 amplified DNA from 5p. Primers DL1 and DL2 amplified a product of similar size for each 5p, 9q, 12p, 15q, 12qter, 18qter.

FIG. 5. Comparison between the sizes of the PCR amplified products after amplification with UN1 and DL2 primers, Lane 1: MW marker; Lane 2: UN1 amplified DNA from 9q; Lane 3. DL2 amplified DNA from 9q; Lane 4: UN1 amplified DNA from 12p; Lane 5: DL2 amplified DNA from 12p; Lane 6: UN1 amplified DNA from 15q; Lane 7: DL2 amplified DNA from 15q; Lane 8: UN1 amplified DNA from 12qter; Lane 9: DL2 amplified DNA from 12qter; Lane 10: UN1 amplified DNA from 18qter; 11: DL2 amplified DNA from 18qter; 12: UN1 amplified DNA from 5p; Lane 13: DL2 amplified DNA from 5p. Primers UN1 and DL2 amplified a product of similar size for each 5p, 9q, 12p, 15q, 12qter, 18qter. UN1 amplified DNA from 12p and 18 qter is in the range of from 300-600 bp.

### V. DETAILED DESCRIPTION OF THE INVENTION

The invention, as described and disclosed herein, provides methods and compositions for detecting a target nucleic acid molecule in a sample. The methods and compositions described herein provide capability for multiple operations to be performed with the utmost accuracy and efficiency. The removed repetitive sequence probes of the invention are highly specific and substantially reduce the background signal or noise that interferes with the detection of the target molecule of interest. The probes prepared using the techniques according to the present invention are of higher quality than those available commercially, and permit significantly faster, more accurate and consistent results.

The methods and compositions of the invention are used in a variety of prognostic, diagnostic, and detection applications. These applications include by way of example and not way of limitation, detection, identification and/or quantification of chromosome abnormalities in mammalian mitotic or interphase cells; detection of genetic diseases, detection of cancer, detection of bacterial or viral infections, detection of biological warfare agents, forensic science, determination of a genetic relationship, such as paternity or species identification, determination of potential donors of organs or tissues, among others.

Definitions

As used herein, “target molecule” refers to a molecule whose presence and/or abundance is being detected. A target can be a whole organism, cellular organelles, or molecules of the organism, or fragments thereof. Most often, a “target molecule” is a polymeric molecule, chromosomes or chromosomal DNA. In preferred embodiments, a “target molecule” is a DNA, RNA, DNA-RNA hybrid, antisense RNA, cDNA, genomic DNA, mRNA, ribozyme, a natural, synthetic, or recombinant nucleic acid molecule, peptide-nucleic acid hybrid, among others. A target molecule can be derived from any of a number of sources, including animals, plants, insects, bacteria, fungi, viruses, and the like. In certain embodiments, the target molecule is a nucleic acid molecule whose sequence structure, presence or absence can be used for certain medical, forensic, or biological warfare detection purposes.

As used herein, “chromosome-specific probe” refers to a combination of detectably labeled polynucleotides that have sequences corresponding to (i.e., essentially the same as) the sequences of DNA from a particular chromosome or sub-chromosomal regions of a particular chromosome (i.e., a chromosome arm). Typically, the chromosome-specific probe is produced by amplification (i.e., using the polymerase chain reaction) of the corresponding chromosomal DNA. A chromosome-specific probe of the invention hybridizes in an essentially uniform pattern along the chromosome or sub-chromosomal region from which it is derived.

As used herein, “chromosomal aberration” or “chromosome abnormality” refers to a deviation between the structure of the subject chromosome or karyotype and a normal (i.e., “non-aberrant”) homologous chromosome or karyotype. The terms “normal” or “non-aberrant,” when referring to chromosomes or karyotypes, refer to the predominate karyotype or banding pattern found in healthy individuals of a particular species and gender. Chromosome abnormalities can be numerical or structural in nature, and include aneuploidy, polyploidy, inversion, translocation, deletion, duplication, and the like. Chromosome abnormalities may be correlated with the presence of a pathological condition and a wide variety of unbalanced chromosomal rearrangements leading to dysmorphology with a predisposition to developing a pathological condition.

As used herein, the term “label” includes molecules that are attached to a nucleic acid molecule of the invention and either alone or in combination with a binding partner assist in the extraction of the repetitive sequences, and/or detection of a hybridization product after hybridization between two nucleic acid molecules of the invention. Most often the label of the invention is a protein-based label, such as biotin, that assists in the extraction of the repetitive sequences with solutions that dissolve and remove proteins.

As used herein, the phrase “molecules attaching a label” refers to molecules that specifically bind to a label molecule and include, for example, any haptenic or antigenic compound such as digoxigenin and anti-digoxigenin; mouse immunoglobulin and goat anti-mouse immunoglobulin, as well as non-immunological binding pairs such as, for example, biotin-avidin, biotin-streptavidin, hormone-hormone receptors, IgG-protein A, and the like.

As disclosed herein, “substantially homologous sequences” include those sequences which have at least about 50%, homology, preferably at least about 60-70%, more preferably at least about 70-80% homology, and most preferably at least about 95% or more homology to another polynucleotide of the invention.

As used herein, “nucleic acid molecule” includes genomic DNA, cDNA, RNA, DNA/RNA hybrid, anti-sense RNA, ribozyme, synthetic forms, and mixed polymers, both sense and antisense strands, and may be chemically or biochemically modified to contain non-natural or derivatized, synthetic, or semi-synthetic nucleotide bases. Also, included within the scope of the invention are alterations of a wild type or synthetic gene, including, but not limited to, deletion, insertion, substitution of one or more nucleotides, or fusion to other polynucleotide sequences, provided that such changes in the primary sequence of the gene do not alter the ability of the nucleic acid molecule to hybridize with the nucleic acid molecule of interest.

The term “sample,” as used herein, includes any sample containing a target nucleic acid molecule that can be detected by composition and methods of the invention. Samples may be obtained from any source including animals, plants, fungi, bacteria, and viruses, among others. Animal samples are obtained, for example from tissue biopsy, blood, hair, buccal scrapes, plasma, serum, skin, ascites, plural effusion, thoracentesis fluid, spinal fluid, lymph fluid, bone marrow, respiratory, intestinal fluid, genital fluid, stool, urine, sputum, tears, saliva, tumors, organs, tissues, samples of in vitro cell culture constituents, fetal cells, placenta cells or amniotic cells and/or fluid.

1. Repetitive Sequences Removed Probes (RSRPs)

In accordance with the present invention, nucleic acid probes are generated in which undesirable repetitive sequences are removed therefrom. The invention generates unique products that are formed after such repetitive sequences have been removed from a source DNA.

In one embodiment, the repetitive sequences removed probe (RSRP) is produced by a method comprising hybridizing source DNA containing both unique and repetitive sequences with driver DNA containing predominately repetitive sequences that hybridize with the repetitive sequences of the source DNA so that the undesirable repetitive sequences of source DNA and the driver DNA hybridize to form a hybridized product. The repetitive sequences of the source DNA, the driver DNA, or both, are attached to a protein-based label moiety that is transferred via hybridization to the hybridized product. The hybridized product containing the repetitive sequences is then extracted with a solution that dissolves or separates proteins from nucleic acid molecules to remove the repetitive sequences from the product. Any protein denaturing solution known to those of skilled within the art may be used in this step of the invention.

In a preferred embodiment, after extraction of the product with a protein denaturing or protein removing solution, the remaining repetitive sequences of the hybridized product are removed by a magnetic force. The product, having a substantial portion of the repetitive sequences removed therefrom, is then recovered by amplification with, for example, PCR using novel unique-sequence primers.

1.1. Source Nucleic Acid Molecules

The source nucleic acid molecules, or source DNA, used in the present invention are microdissected DNAs that are appropriately-selected or synthesized according to the specific target nucleic acid molecule that is to be detected. The source nucleic acid molecule is derived from variety of sources including non-commercial or commercial nucleic acid libraries, including genomic DNA libraries, for example libraries originated from flow-cytometry sorted human chromosomes and cloned DNA fragments (Van Dilla, M. A. et al., Biotechnology, 4:537-552 (1986)); cDNA or RNA libraries, bacterial, and viral genomic or cDNA libraries, artificial chromosome libraries, among others. These libraries are available from several sources including American Type Culture Collection (ATCC).

In one embodiment, the source DNA is a chromosome-specific probe derived from human chromosome DNA libraries. Typically, the chromosome-specific probe is produced by amplification of the corresponding chromosomal DNA. Preferably, the source DNA is amplified by PCR before hybridization with other nucleic acid molecules using a primer such as, for example, degenerate primer UN1(5′CGGGAGATCCGACTCGAGNNNNNNATGTGG-3′) (SEQ ID NO: 1) to directly DOP-PCR (degenerate oligonucleotide-primed) amplify and recover the source DNA.

The human chromosomes libraries include, for example, chromosomes 1, 4, 7, 8, 9, 12, 13, 14, 16, 17, 18, 20, 21, 22, X libraries, or a combination thereof. The chromosome-specific probes are preferably specific for 5p, 9q, 12p, and 15q and chromosome terminal bands 12qter and 18qter. Other libraries include those commercially available under, for example, BD Biosciences Clontech Libraries y, Biocompare Genomic Libraries, Stratagen Human Lambda Genomic Libraries, ATCC Genomic and cDNA Libraries, among others.

In one embodiment, the determination of chromosome abnormalities includes chromosome aberrations, such as those associated with a condition or disease (i.e., deletions, rearrangements, change in chromosome number, etc.) In a preferred embodiment, the chromosome specific probe is a gene probe for leukemia retinoblastoma, human Burkitt's lymphomas, ovarian cancer, uterine cancers, breast cancer or prostate cancer. Chromosome-specific probes hybridize in an essentially uniform pattern along the chromosome or sub-chromosomal region from which it is derived.

Other chromosome or DNA abnormalities are related to, for example, cancer, diseases associated with increased apoptosis including AIDS; neurodegenerative disorders (such as Alzheimer's disease, Parkinson's disease, amyotrophic lateral sclerosis, retinitis pigmentosa, cerebellar degeneration and brain tumor); autoimmune disorders (such as, multiple sclerosis, Sjogren's syndrome, Hashimoto's thyroiditis, biliary cirrhosis, Behcet's disease, Crohn's disease, polymyositis, systemic lupus erythematosus and immune-related glomerulonephritis and rheumatoid arthritis) myelodysplastic syndromes (such as aplastic anemia), graft v. host disease, ischemic injury (such as that caused by myocardial infarction, stroke and reperfusion injury), liver injury (i.e., hepatitis related liver injury, ischemia/reperfusion injury, and cholestosis (bile duct injury).

Cancer includes leukemia such as acute leukemias (i.e., acute lymphocytic leukemia, acute myelocytic leukemia (including myeloblastic, promyelocytic, myelomonocytic, monocytic, and erythroleukemia)) and chronic leukemias (i.e., chronic myelocytic (granulocytic) leukemia and chronic lymphocytic leukemia)), polycythemia vera, lymphomas (i.e., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

The source DNA is appropriately sized in order to facilitate hybridization. In one embodiment, the source DNA is about 1000, 900, 800, 700, 600, 500, 400, 300, 250, 150, 100, 50 or smaller than 50 nucleotides in length. In a preferred embodiment, the source DNA is from about 150 to about 600 nucleotides in length.

1.2. Driver Nucleic Acid Molecules

The invention, as described and disclosed herein, encompasses the use of driver nucleic acid molecules, preferably a driver DNA, that hybridizes to the source DNA and thereby acts to remove hybridization products from ubiquitous repetitive sequences of the source DNA. The driver DNA is selected according to the target nucleic acid molecule being analyzed. For the analysis of human chromosomes, driver DNA is, for example, Cot-1, or total human DNA, which acts to remove from source DNA, via hybridization, ubiquitous repetitive sequences, such as for example, Alu and the KpnI elements.

The total human DNA is available from a variety of sources such as, for example, human genomic DNA from placenta or white blood cells that can be prepared using known techniques, such as that described by Davis et al., Basic methods in molecular biology, Elsevier, N.Y./Amsterdam (1986). The driver DNA is digested or microdisected using standard methods (i.e., with DNAse), to produce driver DNA fragments within the same size distribution as the source DNA.

In one embodiment, the driver nucleic acid molecule additionally contains a carrier DNA from a different source, which carrier DNA competes to hybridize with only a small portion of the human DNA. The carrier DNA is used, as necessary, to adjust the total DNA concentration of the hybridization mixture.

1.3. Labels and Molecules Attaching Labels

Labels are used in the process of making and using repetitive sequences removed probes (RSRPs). In the process of making RSRP, the source DNA, driver DNA, or both are labeled with one or more detectable labels to produce detectably labeled molecules and/or hybridization products.

Several factors govern the choice of labels and molecules attaching labels. including the effect of the label on the rate of hybridization and binding of the nucleic acid fragments to the target DNA, the accessibility of the bound probe to labeling moieties applied after initial hybridization, the mutual compatibility of the labeling moieties, the nature and intensity of the signal generated by the label, the ease of identification and isolation of labeled products, and the like.

Labeled moieties used in the process of making RSRPs preferably include one or more protein-based molecules. Examples of these labels include any haptenic or antigenic compound in combination with an antibody (i.e., digoxigenin and anti-digoxigenin; mouse immunoglobulin and goat anti-mouse immunoglobulin) as well as non-immunological binding pairs (i.e., biotin-avidin, biotin-streptavidin, hormone-hormone receptors, IgG-protein A, and the like).

A more preferred labeling moiety of the invention is a biotin-avidin complex that allows separation and isolation of the labeled molecules via protein separation, as well as enzymatic and magnetic separation techniques (i.e., magnetic beads such as Dynabeads TM; fluorescent dyes). Biotin is particularly useful for several reasons, including the high affinity of avidin and streptavidin for biotin, and the high signal amplification because a large number of biotin molecules can be conjugated to a nucleic acid molecule. The biotinilated source and/or carrier nucleic acid molecules form a biotinilated product that is extracted by a protein denaturing or protein removing solutions such as phenol/chloroform.

Other detectable labels suitable for use in the present invention include any composition detectable by spectroscopic, photochemical, biochemical, immunochemical, electrical, optical or chemical means. These labels include, for example, fluorescent dyes (i.e., fluorescein, fluorescein-isothiocyanate (FITC), Texas red, rhodamine, green fluorescent protein, enhanced green fluorescent protein, lissamine, phycoerythrin, Cy2, Cy3, Cy3.5, Cy5, Cy5.5, Cy7, Fluor X (Amersham), SyBR Green I & II (Molecular Probes); radiolabels (i.e., 3H, 125 I, 35 S, 14 C, or 32 P); enzymes (i.e., hydrolases, particularly phosphatases such as alkaline phosphatase, esterases and glycosidases, or oxidoreductases, particularly peroxidases such as horse radish peroxidase, and the like; substrates; cofactors; inhibitors, chemiluminescent groups; chromogenic agents; and calorimetric labels such as colloidal gold or colored glass or plastic (i.e., polystyrene, polypropylene, latex beads), among others. Patents teaching the use of these labels include U.S. Pat. Nos. 3,817,837; 3,850,752; 3,939,350; 3,996,345; 4,277,437; 4,275,149; and 4,366,241, each of which is incorporated by reference herein in its entirety.

Means of detecting such labels are well known to those of skill in the art. Thus, for example, radiolabels and chemiluminescent labels are detected using, for example, photographic film or scintillation counters. Fluorescent markers are detected using, for example, a photodetector to detect emitted light (i.e., as in fluorescence-activated cell sorting). Enzymatic labels are typically detected by providing the enzyme with a substrate and detecting the reaction product produced by the action of the enzyme on the substrate. Colorimetric labels are detected by simply visualizing the colored label.

In one embodiment, the source DNA, the driver DNA, or both are labeled with biotin, preferably by nick translation (using, for example, Bio-11-dUTP) following standard techniques, such as, Brigati et al., Virology, 126:32-50 (1983); by random primer extension with (i.e., 3′ end tailing), for example, the Amersham multiprime DNA labeling system, substituting dTTP with Bio-11-dUTP.

Also encompassed within the scope of the invention is the use of antibodies as label means. For example, antibodies that specifically recognize RNA/DNA duplexes have been demonstrated to have the ability to recognize probes made from RNA that are bound to DNA targets, Rudkin and Stollar, Nature, 265:472-473 (1977). Antibodies are also used to facilitate visualization of the bound probe wherein the nucleic acid sequences in the probe do not directly carry some modified constituents. Specifically, antibodies to thymidine dimers are reported to be useful for this purpose. Nakane et al., 20 (2):229 (1987), illustrate such a method wherein thyminethymine dimerized DNA (T-T DNA) was used as a marker for in situ hybridization. The hybridized T-T DNA was detected immunohistochemically using rabbit anti-T-T DNA antibody.

In another embodiment of the invention, the bound antibody is detected by detection of a label that becomes associated with the bound antibody after the in situ hybridization is carried out. Detection of the bound antibody may be accomplished in a number of ways. In one embodiment of the invention, the antibody (i.e., the “primary antibody”) is conjugated to a ligand (i.e., biotin). The ligand is then bound in subsequent steps with a detectably labeled anti-ligand, so that the presence of the antigen is detected by the associated label. A wide variety of ligands may be used, and it will be understood that the choice of ligand dictates the subsequent choice of anti-ligand.

In another embodiment of the invention, the “primary” antibody is not conjugated to a ligand and is instead detected using a secondary antibody (i.e., an anti-antibody such as a goat anti-mouse IgG antibody) which is itself labeled or otherwise detectable. In a similar embodiment, a primary antibody bound to antigen is detected by contacting the antibody with detectably labeled protein A or protein G, following the in situ hybridization step. Numerous strategies for amplification or indirect detection of antibodies are known. See, i.e., Ausubel at Chapter 14, and the use of such methods is contemplated in the practice of the present invention.

1.4 Unique PCR Primers

Also encompassed within the scope of the invention is the use of unique primers for recovering, through PCR, the repetitive sequences removed probes (RSRPs). The unique primers are designed based on the nucleic acid sequences of the source DNA probe. Unique primers are designed to increase the specificity of the RSRP for unique sequences in the target nucleic acid molecule without reducing the intensity of binding between the probes and the target nucleic acid molecule. The unique primers of the invention are synthesized, for example, using automated systems well known in the art. Either the entire sequence is synthesized or a series of smaller oligonucleotides are made and subsequently ligated together to yield the full-length sequence.

In one embodiment, the target nucleic acid molecule is a cancer gene and the source DNA is a gene probe for cancer comprising leukemia, retinoblastoma, human Burkitt's lymphomas, ovarian cancer, uterine cancers, prostate cancer, or breast cancer, among others.

In another embodiment, the unique primers comprise DL1, DL2, or a primer that is substantially homologous to D1 or D2. DL1 and DL2 primers share homology with 3′ end and 5′ end of UN1, respectively. Because UN1 primer contains a random hexamer, which may amplify any existing DNA, unique-sequence primers are used in two steps to specifically recover the unique sequences in the source DNA.

The present invention further relates to polynucleotides that hybridize to the herein-described primer sequences. The term “hybridization under stringent conditions” according to the present invention is used as described by Sambrook et al., Molecular Cloning, A Laboratory Manual, Cold Spring Harbor Laboratory Press 1.101-1.104, 1989. Preferably, a stringent hybridization according to the present invention is given when after washing for an hour with 1% SSC and 0.1% SDC at 50° C., preferably at 55° C., more preferably at 62° C., most preferably at 68° C. a positive hybridization signal is still observed. A polynucleotide sequence which hybridizes under such washing conditions with the nucleotide sequence shown in any sequence disclosed herein or with a nucleotide sequence corresponding thereto within the degeneration of the genetic code is a nucleotide sequence according to the invention.

The primers of the invention include polynucleotide sequences that have at least about 50%, 65%, 70%, 75%, 80%, 85%, 90%, 95%, 98%, 99% or more nucleotide sequence identity to the primers. To determine the percent identity of two nucleic acid sequences, the sequences are aligned for optimal comparison purposes (i.e., gaps can be introduced in the sequence of a first nucleic acid sequence for optimal alignment with a second nucleic acid sequence). The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences (i.e., % identity=# of identical overlapping positions/total # of positions×100). In one embodiment, the two sequences are the same length.

The determination of percent identity between two sequences also can be accomplished using a mathematical algorithm. A preferred, non-limiting example of a mathematical algorithm utilized for the comparison of two sequences is the algorithm of Karlin and A ltschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST program of Altschul, et al.,1990, J. Mol. Biol. 215:403-410. BLAST nucleotide searches can be performed with the NBLAST program, score=100, word length=12 to obtain nucleotide sequences homologous to a nucleic acid molecules of the invention. To obtain gapped alignments for comparison purposes, Gapped BLAST can be utilized as described in Altschul et al., 1997, Nucleic Acids Res. 25:3389-3402.

2. In Situ Hybridization

The invention provides novel techniques of hybridizing chromosomes in suspension with fluorescently, or non-fluorescently-labeled RSRPs optionally in combination with flow cytometric analysis or magnetic sorting in order to sensitively, precisely and rapidly quantify a target nucleic acid molecule in a sample.

In one embodiment, the genotypic abnormalities of a sample are determined by in situ hybridization of the RSRPs of the invention that are capable of specifically annealing to one or more sequence in the chromosome or chromosome DNA and detection of the resulting hybrid. In situ hybridization assays are well known and are generally described in Angerer et al., 1987, Methods Enzymol. 152:649-660, Ausubel et al., supra, Pinkel et al., 1988, Proc. Natl. Acad. Sci., 85:9138; Choo, ed., 1994, Methods in Molecular Biology Vol. 33: In situ Hybridization Protocols, Humana Press, Totowa, N.J., each of which incorporated herein by reference in its entirety.

In another embodiment, the in situ hybridization assays according to the invention comprises one or more of the following steps: (1) fixation of the sample chromosome or DNA to be examined, (2) prehybridization treatment of the sample to increase accessibility of target DNA or RNA (i.e., denaturation with heat or alkali), (3) reduce or eliminate nonspecific binding by the use of RSRPs of the invention, (4) hybridization of one or more nucleic acid probes to a nucleic acid molecule in the sample; (5) posthybridization washes and/or nuclease digestion to remove nucleic acid fragments not bound in the hybridization if any; and, (6) amplification and detection of the hybridized target nucleic acid molecules. The reagents used in each of these steps and conditions for their use vary depending on the particular application.

Specifically, as will be appreciated by those skilled in the art, the ambient physiochemical conditions of the target nucleic acid molecule and the RSRP for particular applications can be adjusted by controlling several factors, including, inter alia, concentration of the constituents, incubation time of the target nucleic acid molecule in the solution and the concentrations, complexities, and lengths of the RSRPs. For example, the total concentration of the chromosomes or chromosomal DNA in the hybridization mixture has a concentration range of about 0.1, 0.5, 1, 2, 3, 4, 5, ug/ul and RSRPs have a concentration range of about 1, 10, 15, 20, 30, 40, or 50 ng/ul are used. Preferably, about 1 ug/ul of the whole chromosome or chromosomal DNA was used with 20 ng/ul of RSRP. The hybridization conditions must be sufficiently close to the melting temperature to minimize non-specific binding. On the other hand, the conditions cannot be so stringent as to reduce correct hybridizations of complementary sequences below detectable levels.

2.1. In situ Solution Hybridization (ISSH)

The invention provides methods and probes for detecting and quantifying nucleic acid molecules, including whole chromosomes in solution. The hybridization technology of the invention may be used in a DNA chip format for high throughput screening purposes. The in situ solution hybridization (ISSH) method of the invention provides improvements over the traditional in situ hybridization and solution hybridization techniques described in U.S. Pat. No. 6,077,671, incorporated herein by reference in its entirety. The hybridization methods of the invention are based on probes that allow both capture of target nucleic acids and quantification of the captured targets. The technologies are particularly useful for identifying and quantifying chromosomal rearrangements and deletions that are characteristic of many hematological malignancies, solid tumors, ionizing radiation, or other environmental agents on the frequency of chromosome aberrations.

The ISSH technology, according to the invention described herein, enables in situ hybridization on various numbers of isolated individual chromosomes in suspension and offers the possibility of sorting chromosomes based on FISH signals and bulk detection of chromosomal exchange rearrangements. Prior to the current invention, attempts to perform in situ hybridization on chromosomes in solution have been hindered by chromosome loss, breakage, and aggregation (Bao-Tram et al. 1995, Kraus et al. 1995). The invention described herein provides several steps for substantially reducing chromosome loss and chromosome clumps, while preserving chromosome morphology.

ISSH generally provides two additional steps with regard to standard isolation and in situ hybridization of chromosomes in order to reduce chromosome loss so that a large number of good quality metaphase chromosomes are obtained and hybridized. In the first step, chromosomes treatment with RNase decreases cell debris and removes residual RNA from the target. Such removal can be accomplished by, for example, incubation of the chromosomes or fixed chromosomes in 50-100 microgram/milliliter RNase in 2×SSC (where SSC is a solution of about 0.15M NaCl and about 0.015M sodium citrate) for a period of 1-2 hours at room temperature.)

In the second step, the chromosomes are fixed prior to hybridization. Fixatives include, for example, acid alcohol solutions, acid acetone solutions, Petrunkewitsch's reagent, and various aldehydes such as formaldehyde, paraformaldehyde, glutaraldehyde, or the like. For cells or chromosomes in suspension, a fixation procedure is disclosed by Trask, et al., Science: 230, 1401-1402 (1985) and Trask et al., Hum. Genet: 78:251-259 (1988), each of which is incorporated herein by reference in its entirety. In a more preferred embodiment of the invention, the fixative agent is a 3:1 solution of methanol:acetic acid, which is used prior to hybridization.

Also included within the scope of the invention are ISSH techniques that require pre-hybridization treatment of chromosomes with agents to remove proteins. Such agents include enzymes or mild acids. Pronase, pepsin or proteinase K are frequently used enzymes. A representative acid treatment is 0.02-0.2 N HCl, followed by high temperature (i.e., 70° C. washes). Optimization of deproteinization requires a combination of protease concentration and digestion time that maximizes hybridization, but does not cause unacceptable loss of morphological detail. Optimum conditions vary according to tissue types and method of fixation. Additional fixation after protease treatment are also included within the scope of the invention. Thus, for particular applications, some experimentation is required to optimize protease treatment.

In order to reduce the viscosity of the chromosome solution (i.e., chromosomes in hybridization buffer), the solution is diluted, for example by mixing 1:1 with a solution containing, for example, about 80 mL of 0.15 M NaCl/0.015M Na citrate mixed with about 20 mL of double distilled water, pH 7 before spinning down the chromosomes. Hybridization buffers commonly contain a high concentration of dextran sulfate (10% dextran sulfate is standardly used in hybridization buffers) which causes the hybridization buffer to be highly viscous. It is believed that the high level viscosity of standard hybridization solutions cause chromosomes to be retained in solution and thus lost during centrifugation. By diluting the hybridization buffer before centrifugation (or by using a more dilute hybridization buffer), the viscosity of the hybridization solution is decreased, thereby lessening the drag on chromosomes during centrifugation and allowing more chromosomes to spin down. As a result, a higher percentage of the chromosomes in solution are recovered. The final concentration of dextran sulfate in the chromosome solution before spin down is preferably less than about 10% and preferably less that about 5% and can be decreased even further.

In another embodiment, ISSH includes hybridization of chromosomes in a diluted hybridization buffer, for example, 40% formamide without 10% dextran sulfate. Diluting the hybridization buffer and deleting 10% dextran sulfate decreased the viscosity of the buffer compared with the conventional hybridization buffer with the high concentration (70%) of formamide and 10% concentration of dextran sulfate. This method lessened the drag on the chromosomes during centrifugation, allowing more chromosomes to spin down and preserving better morphology.

A low recovery of chromosomes after FISH in the prior art suspensions compromised chromosome aberration analysis and was inferior to the hybridization on slides. The recovery of chromosomes following prior art hybridization in suspension was evaluated and compared with the recovery of chromosomes following the method of invention (ISSH). Chromosome recovery, after hybridization in suspension and washes by the method described herein ranged from 46% to 73% (Table 1). Table 1 shows the reproducibility of the method of the invention to recover large numbers of chromosomes after hybridization. As shown in Table 1, the chromosome recovery was routinely over 60%. Similar results were obtained for solution-hybridization of the human cell line, GM 130B, and the hamster×human hybrid cell line.

Because previous studies (Dutdin et al. 1987; Dutdin et al. 1988) did not provide quantitative information on chromosome loss, the hybridization method disclosed in these studies were performed side by side with the hybridization method of the invention, in order to compare the chromosome recovery. Both methods were performed in parallel on aliquots of the same chromosome suspension. In this comparative study, 62.9% of chromosomes were recovered using the method of the invention, as compared with 4.6% using the Dudin method (Table 2). The high chromosome recovery by the method of invention suggests that ISSH provide a practical tool for bulk analysis of chromosome aberrations.

TABLE 1
CHROMOSOMES RECOVERED AFTER SUSPENSION HYBRIDIZATION
AS DETERMINED BY HEMOCYTOMETRIC METHODS

| | | NUMBER OF INDIVIDUAL | PERCENT |
|---|---|---|---|
| NO. OF | NO. OF CHROMOSOMES | CHROMOSOMES RECOVERED | CHROMOSOME |
| SAMPLES | BEFORE HYBRIDIZATION | POST HYBRIDIZATION | RECOVERY |
| 1 | 4.0 × 10⁵ | 2.3 × 10⁵ | 57.5% |
| 2 | 1.5 × 10⁶ | 8.8 × 10⁵ | 58.7% |
| 3 | 1.5 × 10⁶ | 9.1 × 10⁵ | 60.7% |
| 4 | 1.5 × 10⁵ | 1.0 × 10⁶ | 66.7% |
| 5 | 1.5 × 10⁶ | 1.1 × 10⁶ | 73.3% |
| 6 | 3.5 × 10⁶ | 2.2 × 10⁶ | 62.9% |
| 7 | 3.5 × 10⁶ | 2.4 × 10⁶ | 68.6% |
| 8 | 3.5 × 10⁶ | 1.6 × 10⁶ | 45.7% |
| 9 | 3.5 × 10⁶ | 2.1 × 10⁶ | 60.0% |

TABLE 2
CHROMOSOMES RECOVERED AFTER SUSPENSION HYBRIDIZATION,
AS DETERMINED BY FLUORESCENCE MICROSCOPY

| | NUMBER OF CHROMOSOMES | | |
|---|---|---|---|
| | | RECOVERED | |
| | BEFORE | AFTER | |
| METHOD | HYBRIDIZATION | HYBRIDIZATION | RECOVERY |
| Hamster × human cell line | | | |
| Dudin et al. 1987¹ | 3.5 × 10⁶ | 1.6 × 10⁵ | 4.6% |
| Preliminary study² | 3.5 × 10⁶ | 2.2 × 10⁶ | 62.9% |
| Human GM130B cell line | | | |
| Preliminary study² | 4.0 × 10⁶ | 2.5 × 10⁶ | 62.5% |

¹Data generated in our lab using the method of Dudin et al. 1987.
²Preliminary study using our method (He et al. 2001).

Using ISSH, it is possible to specifically stain or label any selected individual chromosome (or chromosomes) referred to as a target chromosome, or a subregion or fragment thereof. The present method has also been shown to be useful in a variety of cells, both in mitotic (i.e., metaphase, prophase) and interphase cells. For example, ISSH is used for rapidly screening mitotic and interphase aneuploid tumor cells for complex numerical and structural aberrations of individual chromosomes (i.e., changes in number of chromosomes, deletions and rearrangements or translocations). ISSH is also used to identify chromosome-specific sequences and, subsequently, to separate them from repetitive sequences.

3. Application of ISSH and RSRP In The Early Detection of Cancer

The hybridization methods and probes of the invention greatly enhance early detection of minimal residual malignant cells in bone marrow, lymph nodes and peripheral blood in cancer patients. Several types of cancers, such as leukemias and lymphomas, are genetic disorders by nature. Each genetic alteration, whether an initiating or a progression-associated event, may be mediated through gross chromosome changes and therefore has the potential to be cytogenetically visible. It is well known that specific chromosomal translocations play important roles in hematological malignancies (Yunis, 1983; Solomon et al. 1991, Rabbitts, 1994; Sandberg and Chen, 1995).

Minimal residual disease (MRD) of leukemias and lymphomas has been a major problem in cancer therapy. The detection of cancer-specific chromosome translocations in bone marrow, lymph nodes, and peripheral blood, is a promising way for early detection of MRD. It is widely believed that early detection of MRD increases patient's survival and profoundly impacts cancer patient's management. Morphologic examination of bone marrow, however, detects the presence of malignant cells with a low sensitivity (1×100). Additionally, measuring chromosome translocations is very labor intensive and slow, by conventional methods. Although there are commercial products using PCR to test for MRD, the PCR method is not sufficiently quantitative to permit accurate measurement of the frequency of chromosome rearrangements.

The invention, as disclosed and described herein, provides novel techniques of hybridizing chromosomes from cancer patients with leukemias or lymphomas in both pre- and post-therapy via in situ solution hybridization and the use of the RSRPs of the invention. The hybridization method and probes of the invention sensitively, precisely and rapidly quantify cancer-related chromosome translocations by bulk analysis and quantitatively measure the frequency of cytogenetic markers associated with specific cancers. Hybridizing chromosomes in suspension according the method of the invention has a sensitivity of approximately 1×1,000,000.

4. Application of RSRP In The Early Detection of Biological Weapon Agents

Also encompassed within the scope of the invention are methods and compositions for rapid and accurate detection of minute amounts of biological warfare agent within various media of dissemination. The biological warfare agents include, for example, Bacillus anthracis, Botulinum toxin, Plague, Smallpox, Francisella tularensis, Hemorrhagic Fever Viruses (HFVs), Trichothecene mycotoxins, among others.

In one embodiment, RSRPs specific to detect anthrax are provided. Anthrax source DNA is, for example, a gene probe of anthrax derived from Bacillus anthracis, Bacillus cereus or Bacillus Thuringiensisn. The carrier DNA is, for example, a genomic DNA of anthrax containing repetitive ubiquitous sequences, microdissected and labeled with a label moiety such as biotin. Hybridization of the source DNA and carrier DNA of anthrax results in the production of a biotinilated product. The biotinylated product is then extracted one or more times with phenol/chloroform and the resulting product is subjected to a magnetic separation using avidin-coated magnetic beads. The product is recovered by PCR using anthrax-specific primers set forth below. PCR primer sets from different strains of anthrax, as shown in Table 3 below, are used as described in Jackson et al., Proc. Natl. Aca. Sci. U.S. 95:1224-1229 (1998), incorporated herein by reference in its entirety.

TABLE 3
PRIMER SETS FOR IDENTIFICATION OF SEVERAL ANTHRAX GENES

| | | | | | |
|---|---|---|---|---|---|
| PRIMER | | GENE/ | | | AMPLICON |
| SET | P/N | ACCESSION NO. | LOCATION | PRIMER SEQUENCE | SIZE, BP |

| | | | | | | |
|---|---|---|---|---|---|---|
| GPR-4 | P | | | ACAACTACCACCGATGGC | (SEQ ID NO: 4) | |
| GPR-5 | P | VrrA/L48553 | C | TTATTTATCATATTAGTTGGATTCG | (SEQ ID NO: 5) | 377-425 |
| EWA-1 | N | | | TATGGTTGGTATTGCTG | (SEQ ID NO: 6) | |
| EWA-2 | N | VrrA/L48553 | C | ATGGTTCCGCCTTATCG | (SEQ ID NO: 7) | 142-190 |
| PA-1F | P | | | CCAGACCGTGACAATGATG | (SEQ ID NO: 8) | |
| PA-1R | P | Pag/M22589 | PX01 | CAAGTTCTTTCCCCTGCTA | (SEQ ID NO: 9) | 508 |
| PA-2F | N | | | CGAAAAGGTTACAGGACGG | (SEQ ID NO: 10) | |
| PA-1R | N | Pag/M22589 | PX01 | CAAGTTCTTTCCCCTGCTA | (SEQ ID NO: 11) | 409 |

5. Test Kits

Test kits are used to detect a target nucleic acid molecule in a sample.

In one embodiment, the test kit is used for the diagnosis, identification, detection and/or quantification of a chromosome or chromosome region of interest (i.e., one which is associated with a genetic disorder or causes an infectious disease). Such test kits can be made to include one or more RSRPs having chromosome-specific sequences derived from one or more chromosome(s) of interest.

In another embodiment, RSRPs include chromosome-specific sequences from chromosomes 1, 4, 7, 8, 9, 12, 13, 14, 16, 17, 18, 20, 21, 22, X, or a combination thereof. Preferably the chromosome specific sequences are derived from chromosomes regions 5p, 9q, 12p, and 1Sq and chromosome terminal bands 12qter and 18qter, among others. In a preferred embodiment, the test kit is used to detect cancer such as leukemia or lymphomas.

In yet another embodiment, the test kit is used to detect biological agents, such as, for example, biological warfare agents, in a patient's sample or in the environment. In this embodiment, the RSRPs are made to include a biological warfare agent's unique and specific sequence(s).

Generally, the reagents and devices described herein are packaged to include any if not all of the necessary components for performing the various applications of detection of nucleic acid molecules described herein. For example, the kits can include any of templates, buffers, other chemical agents, nucleotides, control materials, devices, or the like. Such kits also typically include appropriate instructions for using the devices and/or reagents. Generally, reagents are provided in a stabilized form, so as to prevent degradation or other loss during prolonged storage, i.e., from leakage.

This invention is further illustrated by the following examples, which are provided by way of illustration only and are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims. Those of skill will readily recognize a variety of non-critical parameters, which are changed or modified to yield essentially similar results.

### EXAMPLES

### Example 1

### PCR Amplification of Source DNA and Preparation of Biotin Labeled Driver DNA

The degenerate primer UN1 (5′CGGGAGATCCGACTCGAGNNNNNNA TGTGG-3′) (SEQ ID NO: 1) was first used to directly DOP-PCR and recover the source selected microdissected DNA fragments. 2 μl of each selected chromosome DNA was added to PCR reaction mix (50 μl) which contains 10 mM Tris-HCl, pH 8.4, 2 mM MgCl2, 50 mM KCl, 200 μM each dNTP, 2 μM primer and 2 units Taq DNA polymerase. The reaction was heated to 96° C. for 2 min, followed by 25 cycles at 94° C. for 1 min, 1 min at 56° C., and 1 min at 72° C., with a 5-min final extension at 72° C.

A driver DNA was created as follows: human genomic DNA that predominantly contains repetitive sequences was microdisected and biotin-labeled. The mixture of driver DNA was labeled with biotin by nick translation. For example, 5 μl of 10×dNTPs including biotin-16-dUTP were mixed with 3 μg driver DNA and 5 μl DNA Polymerase I/DNase I in a total volume of 50 μl, then incubated at 16° C. for 6 hours.

### Example 2

### Hybridization of Driver DNA to Source DNA

Driver DNA (10 μg) was labeled with biotin by nick translation. After amplification with the UN1 primer, 100 ng microdissected source DNA was hybridized with 10 μg biotin-labeled human repetitive sequences, i.e., driver DNA, in 20 μl hybridization solution (6×SSC, 0.2% SDS) at 55° C. overnight. After hybridization, 20 μl Avidin (5 μg/ml)(Vector Laboratories, Inc., CA) was added to the hybridization mix and incubated at 37° C. for 20 min.
a) First Subtraction of repetitive Sequences from Source DNA

After incubation of the driver DNA and source DNA as described above, 240 μl ddH20 and 300 μl buffer saturated phenol were added to the hybridization mixture, vortexed for 30 sec, and centrifuged at 14,000 rpm for 5 min. The supernatant was transferred to a clean tube with 300 μl Phenol:chloroform:Isoamyl Alcohol (25:24:1), vortexed for 30 sec, and centrifuged at 14,000 rpm for 5 min.

b) Precipitation of Supernatant

The supernatant was transferred to a clean tube with chloroform, vortexed for 30 sec and centrifuged a t 14,000 rpm for 5 min again. The supernatant was then transferred to a clean tube and 1/10 volume of 3M Sodium Acetate and 2.5 volume 100% EtOH were added, mixed and precipitated at −20° C. overnight. The tube was centrifuged at 14,000 rpm for 30 min, the supernatant was discarded, the pellet air dried, and re-suspended in 10 μl dH20.
c) Recovery of Precipitated Product by PCR

Because UNI primer contains a random hexamer, which may amplify any existing DNA, unique-sequence primers were used in two steps to specifically recover the source DNA (i.e,. the precipitated product) post the above phenol subtraction. First, the primer DL1(5′TTCACTGATACCGACTCGAGNNNNNNATGTGG-3′) (SEQ ID NO: 2) was used. This primer shares homology with the 3′-end of UN1. The reaction was cycled 5 times at 94° C. for 1 min, 50° C. for 1 min, and 72° C. for 1 min and then 24 cycles at 94° C. for 1 min, 60° C. for 1 min, and 72° C. for 1 min, with the final extension at 72° C. for 5 min. Second, the primer DL2 (5′-TTCACTGATACCGACTCGAG-3′) (SEQ ID NO: 3) was used. This primer shares a unique sequence of 20 bases at the 5′-end with DL1. The reaction was cycled 20 times at 94° C. for 1 min, 56° C. for 1 min, and 72° C. for 1 min, with the final extension at 72° C. for 5 min.
d) Re-hybridization to Driver DNA

The above recovered source DNA was reacted with biotin-labeled driver DNA again following the same hybridization procedure as detailed above for the Hybridization to source DNA.

### Example 4

### Subtraction of Repetitive Sequences from Source DNA by Magnetic Beads Post Ethelol Subtraction

Method 1: The source DNA obtained in example 3 was purified further as follows.

(1) Cool the two hybridized DNA (source DNA and driver DNA) to room temperature.

(2) 4.4 mg (440 p1) streptavidin magnetic particles (Boehringer Mannheim) were prepared according to the manufacturer's instructions and resuspended in 125 μl of 10 mM TRIS-HCl, pH 8.0, 1 mM EDTA, pH 8.0, 2 M NaCl (2× binding and washing buffer). 100 μl streptavidin magnetic particles were added to 100 μl hybridized DNA mixture and incubated at room temperature for 30 min with shaking. Tubes were then applied to a magnetic particle separator (Boehringer Mannheim) for 3 min and the supernatant was gently removed. This supernatant was added directly to the remaining, unused magnetic particles with buffer freshly removed, and incubated with axial rotation as above. The second supernatant (200 μl) was removed and DNA purified using a QIAex II kit (Qiagen) according to the manufacturer's instructions, and resuspended in 25 μl TE (10 mM TRIS-HCl, pH 8.0, 1 mM EDTA, pH 8.0).

Method 2: Alternatively, after cooling the DNAs were added to an equal volume of 2×ALTech's binding buffer # 5 (1 M NaCl, PNM plus 2% BSA). Streptavidin coated magnetic beads (4.4 mg (440 μl)) were added and incubated at 42° C. for 2-3 hours with slight shaking. The ALTech's binding buffer #5 facilitates attachment of biotin-labeled DNA to the magnetic beads with minimum DNA to DNA sticking, and with minimum attachment of non-hybridized DNA to the beads. The beads were then concentrated using a magnet.

Purified and highly selected source DNA after the two above-described subtractions was recovered with PCR amplification using unique-sequence primers, i.e., DL1 (firstly) and DL2 (secondly). The procedure for recovery of the probe was the same as described in example 2 above. The final resulting removed repetitive sequence source DNA were labeled directly with fluorochromes or indirectly with a hapten (such as biotin or digoxigenin) and were used as a DNA probe for genetic abnormalities.

### Example 5

### Phenol Subtraction Procedure

To determine if the sole phenol subtraction procedure efficiently removes avidin-biotin complexes, the following experiment was performed:
(1) Incubations: 2 μg biotin-labeled driver DNA in 20 μl hybridization solution (6×SSC) was denatured in 98° C. for 5 min, 40 μl ddH20 and 3 μl avidin was added and incubated at 37° C. for 20 min, 0.3 μl anti-avidin DCS-F was added and incubated at 37° C. for 20 min.

(2) Subtraction: 240 μl ddH20 and 300 μl buffer saturated phenol were added to the hybridization mixture, vortexed for 30 sec, and centrifuged at 14,000 rpm for 5 min. The supernatant was transferred to a clean tube with 300 μl of Phenol: chloroform:Isoamyl Alcohol (25:24:1), vortexed for 30 sec, and centrifuged at 14,000 rpm for 5 min.

(3) Precipitation of Supernatant: The supernatant was transferred to a clean tube with chloroform, vortexed for 30 sec and centrifuged at 14,000 rpm for 5 min again. The supernatant was transferred to a clean tube and 1/10 volume of 3M Sodium Acetate and 2.5 volume 100% EtOH were added, mixed well and precipitated at −20° C. overnight. The tube was centrifuged at 14,000 rpm for 30 min, the supernatant was discarded and the pellet was air dried and resuspended in 10111 dH₂O and 5111 was electropheresed on a 1% agarose gel.

(4) The DNA fragments obtained after the first round of subtraction were put through a 2nd round of phenol subtraction.

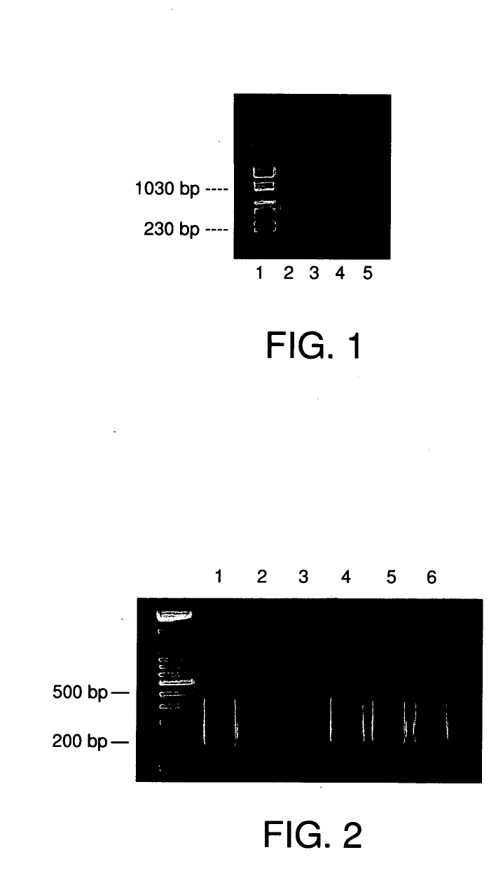

(5) Analysis of precipitated product: The DNA before and after subtraction was electrophoresed on an agarose gel (FIG. 1, lane 1). Before subtraction, a smear of 200-1000 bp DNA was observed (lane 1). The precipitated product after the 1^{st} and 2^{nd} subtraction was compared to the sample before subtraction. As shown in FIG. 1, the majority of biotin-labeled driver DNA was removed after the 1^{st} subtraction (lane 2). This indicated that the phenol subtraction method used was efficient for removing avidin-biotin complexes.

### Example 6

### Recovery of 15q Probe after the Phenol Subtraction Procedure

In order to assure that the unique sequences were not removed during the subtraction process and could readily be recovered from the supernatant using the DL2 unique primer. The following experiment was performed.

(1) Incubation: 100 ng microdissected DNA of chromosome15q (either biotinylated or unlabeled) was mixed with 20 μl hybridization solution. Avidin, and anti-avidin were added as described above and incubated at 37° C. for 20 min.

(2) Subtraction and precipitation: The same subtraction and precipitation methods were applied as described above. The DNA fragments obtained after the first round of subtraction were put through a 2nd round of phenol subtraction.

(3) Recovery of microdissected DNA by PCR: Microdissected DNA fragments were recovered by PCR using DL2 primer. The PCR reaction was cycled 25 times at 94° C. for 1 min, 64° C. for 1 min, 72° C. for 1 min, with the final extension at 72° C. for 5 min, and product was precipitated.

(4) Evaluation of the recovered RSRPs. The PCR product was evaluated on a 1% agarose gel. When biotinylated microdissected DNA (FIG. 2, lane 1) was used in the subtraction procedure, the added avidin bound to the DNA, and the majority of the DNA was removed after the first round of subtraction (lane 2). When non-labeled microdissected DNA (lane 4) was used in the procedure, the added avidin did not have anything to bind to. The DNA was efficiently recovered after the first round of subtraction (lane 5) and after the second round of subtraction (lane 6). These results demonstrated the feasibility of recovering the non-labeled microdissected DNA following multiple rounds of subtraction. The results of the two preliminary tests demonstrated that the phenol subtraction itself was reasonably efficient for the subtraction of repetitive sequences and optimal for the recovery of unique sequences in source DNA using the DL2 primer.

### Example 7

### Subtraction of Repetitive Sequences from Microdissected and DL2-Amplified DNA from 5p, 9q, 12p, 15q, 12qter, and 18qter and Recovery of Unique Sequences.

(1) Hybridization: In separate subtraction reactions, 100ng microdissected DL2 amplified DNA from 5p, 9q, 12p, 15q, 12qter, and 18qter were mixed with biotin-labeled driver DNA in 2011 hybridization solution. The mixture was denatured in 98° C. for 5 min. 40 μl ddH20 and 311 Avidin were added and incubated at 37° C. for 20 min, 0.311 anti-avidin DCS-F was added and incubated at 37° C. for 20 min.

(2) Subtraction: Subtraction and precipitation were performed as described above and repeated 2-3 times.

(3) Recovery of microdissected DNA: Microdissected DNA from 5p, 9q, 12p, 15q, 12qter, and 18qter were recovered by PCR using the DL2 primer as described above.

(4) Labeling with Digoxygenin: The resulting repetitive-sequence removed probes were labeled with digoxygenin and purified using a PCR purification kit.

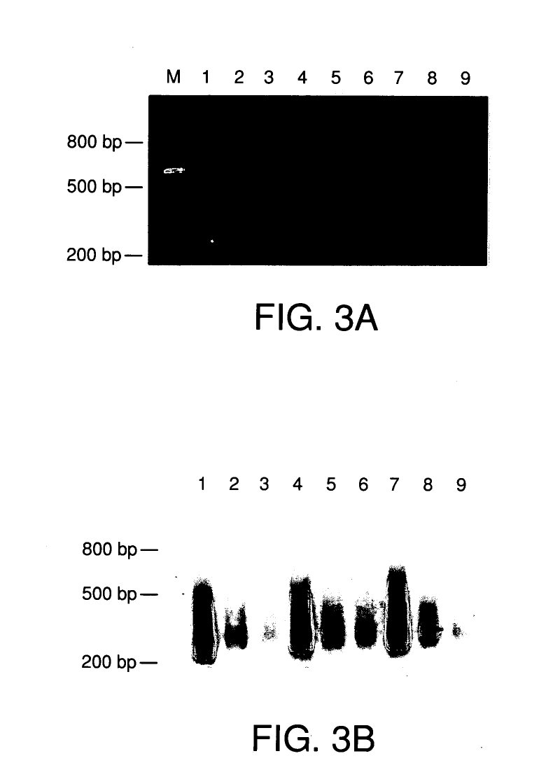

(5) Assessment of the quality of recovered DNA sequences: To determine if the repetitive sequences were successfully removed southern blot analysis was performed. Equal amounts of microdissected DNA probes were loaded in each well and run on a 1% agarose gel (FIG. 3A). The gel was denatured and neutralized. The DNA was transferred to a nylon membrane by alkaline capillary blotting, fixed by cross-linking, and hybridized with 32P labeled Cot-1 DNA overnight at 65° C. The membranes were washed using routine procedures and exposed for 6 hours. Microdissected DNA of 9q, 12p, and 15q before subtraction are shown in lanes 1, 4, and 7, respectively, and after the first round in lanes 2, 5, and 8, respectively, and second round of subtraction in lanes 3, 6, and 9, respectively. The hybridization results demonstrated the efficient removal of the repetitive elements. As shown in FIG. 3B, the majority of the repetitive sequences were removed after the first round of subtraction.

### Example 8

### Evaluation of Amplified Products

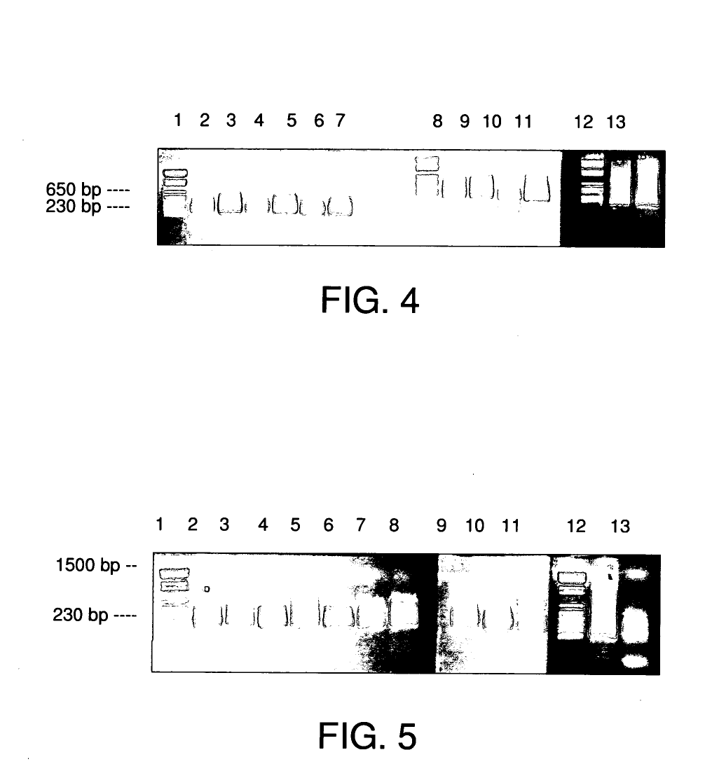

The size of the DNA after each amplification step was compared on an agarose gel. Recovered PCR amplified products using UN1, DL1, or DL2 primers were analyzed on an agarose gel and the size of DNA of each product was compared as shown in FIGS. 4 and 5. FIG. 4 shows a comparison between the sizes of the PCR amplified products after amplification with DL1 and DL2 primers. FIG. 5 shows a comparison between the size of the PCR amplified products after amplification with UN1 and DL2. As shown in FIGS. 4 and 5, primers UN1, DL1 and DL2 amplified a product of similar size for each 5p, 9q, 12p, 15q, 12qter, 18qter. The size of DNA from UN1 amplified DNAs of 12p and 18qter were about 300-600 bp. The variation in the size of the DNA did not affect the FISH results.

### Example 9

### Assessment of the Quality of Removed Repetitive Sequence Probes (RSRPS)

FISH was performed on metaphase chromosomes using Dig-labeled repetitive-sequence removed probes 5p, 9q, 12p, 15q, 12qter and 18qter. Pretreatment with Cot-1 DNA in the hybridization procedures was not performed. The hybridization was performed under 45° C. The results were assessed following half hour, 1 hour, 3 hours of hybridization and also by the standard overnight hybridization.

FISH results on metaphase chromosomes: In all the hybridization cases, the painting signals were clearly visible, uniform, and bright, with little to no background staining. Furthermore, the unique sequences isolated were specific to each region and did not cross react with any other regions of the chromosome. The repetitive sequence probes advantageously did not require an overnight hybridization for obtaining good staining. Comparable results were obtained with hybridization of about 30 minutes. The FISH results demonstrated that comparable results were obtained using non-sequence depleted probes and Cot-1 during the hybridization process. These results showed that the sole phenol subtraction was capable of removing most of the repetitive sequences from the microdissected DNA, as no Cot-1 or pre-annealing was required to generate ideal staining in these experiments.

All references discussed herein are incorporated by reference. One skilled in the art will readily appreciate that the present invention is well adapted to carry out the objects and obtain the ends and advantages mentioned, as well as those inherent therein. The present invention may be embodied in other specific forms without departing from the spirit or essential attributes thereof and, accordingly, reference should be made to the appended claims, rather than to the foregoing specification, as indicating the scope of the invention.

BIBLIOGRAPHY

Bao-Tram, N., K. Lazzari, K. and Abebe, J. In situ hybridization to chromosomes stabilized in gel microdrops. Cytometry, 1995, 21, 111-119.
Chen, Z., Meloni-Ehrig, A., Shepard, R., Palumbos, J. C., Guan, X. Y., Carroll, K. L., Dent, K. M., Carey, J. C., Pure trisomy 10p resulting from an extra ring chromosome: characterization by advanced molecular cytogenetics. Am. J. Med Genet., 2001, 102, 379-382.
Craig, J. M., Kraus, J., Cremer, T., Removal of repetitive sequences from FISH using PCR-assisted affinity chromatography. Human Genetics, 1997, 100(3-4), 472-476.
Dudin, G., Cremer, T., Schardin, M., Hausmann, M., Bier F. and Cremer, C., A method for nucleic acid hybridization to isolated chromosomes in suspension. Human Genetics, 1987, 76, 290-292.
Dudin, G., Steegmayer, E. W., Vogt, P., Schnitzer, H., Diaz, E., Howell, K. E., Cremer, T. and Cremer, C., Sorting of chromosomes by magnetic separation. Human Genetics, 1988, 80, 111-116.
Durm, M., Schussler, L., Munch, H., Craig, J., Ludwig, H., Hausmann, M., Cremer, C., Fast-painting of human metaphase spreads using a chromosome-specific, repeat-depleted DNA library probe. Biotechniques, 1998. 24 (5), 820-825.
Guan, et al., Genomics, 22(1):101-107 (1994)), (Guan et al., Nature Genet 12: 10-11 (1996)) chromosome arm panting probes (CAPs)
Guan et al., Clinic Cancer Res., 1: 11-18 (1995)) and chromosome band-specific painting probes.
He, H., Deng, W., Cassel, M., Lucas, J. N., Isolation and Fluorescence In situ Hybridization of Metaphase Chromosomes in Suspension. International Journal of Radiation Biology, 2001, 77, 787-795.
Kraus, M., Hausmann, M., Hartig, R., Durm, M., Haar, F. M. and Cremer, C., Non-encymatic, low temperature in situ hybridization of metaphase chromosomes for magnetic labelling and sorting. Experimental Technique of Physics Experimentelle Technik der Physik, 1995,41,139-153.
Licheter, P., Cremer, T., Tang, C.-J., Watkins, P., Manuelidis, L., Ward, D., Rapid detection of human chromosome 21 aberrations by in situ hybridizaion. PNAS USA, 1988, 85, 9664-9668.
Lucas, J. N., Swansbury, G., Clutterbuck, R., Hill, F., Burk, C., and Straume, T., Discrimination between leukemia- and non-leukemia-induced chromosomal abnormalities in the patient's lymphocytes. International Journal of Radiation
Pinkel, D., Landegent, J., Collins, C., Fuscoe, J., Segraves, R., Lucas, J., Gray, J. Fluorescence in situ hybridization with human chromosome-specific libraries: detection of trisomy 21 and translocations of chromosome 4. PNAS USA, 1988, 85, 9138-42.
Rabbitts, T. H. Chromosome translocations in human cancer, Nature, 1994, 372(10), 143-149.
Solomon, E., Borrow, J. and Goddard, A., Chromosome aberrations and cancer, Science, 1991, 254, 1153-1159.
Sandberg, A. A., Chen, Z. (1995): Cytogenetics of Acute Leukemia.In:Neoplastic Diseases of the Blood, 3rd ed. Wiernik P H, Canellos G P, Dutcher J P, Kyle R A, eds. Churchill Livingstone.
Sandberg, A. A., Chen, Z. (2000): FISH Analysis. In: Methods in Molecular Medicine, Vol. 55: Hematologic Malignancies: Methods and Techniques. Faguet G B, eds. Humana Press, Inc.
Yunis, J. J., The chromosome basis of human neoplasia. Science, 1983, 221, 227-236.
Miteiman et al., Human Gene Mapping, Cytogenetic Cell Genet, 58:653-79 (1991))
Frezal et al., Human Gene Mapping II, Cytogenetic Cell Genet, 58:986-1052 (1991))
Yunis and Ramsay, Retinoblastoma and Subband Deletion of Chromosome 13, Am. J. Dis. Child. 132:161-163 (1978))
Sandberg, The Chromosomes In Human Cancer And Leukemia, Elsevier; New York, pp.10 (1990)).
Zech, et al., Int. J. Cancer 17:47-56 (1976)).

1. A method of producing a probe having removed repetitive sequences comprising:
(a) providing a source nucleic acid molecule containing repetitive sequences;
(b) providing a driver nucleic acid molecule attached to a label and containing repetitive sequences that hybridize with the repetitive sequences of the source nucleic acid molecule;
(c) hybridizing the source nucleic acid molecule and the driver nucleic acid molecule in the presence of a molecule that binds the label of step (b) wherein the repetitive sequences of the source nucleic acid molecule hybridize with the repetitive sequences of the driver nucleic acid molecule to form a product;
(d) subtracting the hybridized repetitive sequences of the product of step (c) by extraction with a protein dissolving solution to remove the hybridized repetitive sequences from the product; and
(e) recovering the probe having repetitive sequences removed therefrom.

2. The method of claim 1, wherein the recovering step (e) is performed by PCR with unique-sequence primers.

3. The method of claim 2, wherein the unique sequences comprise DL1 and DL2, respectively.

4. The method of claim 1, wherein the recovered probe of step (e) is processed one or more times through steps (a) to (e).

5. The method of claim 1, wherein the source nucleic acid molecule, the driver nucleic acid molecule, or both are attached to a label.

6. The method of claim 1, wherein the label is biotin and the molecule that attaches the label is avidin.

7. The method of claim 1, wherein the extraction of step (d) is performed by phenol/chloroform.

8. The method of claim 7, wherein the molecule that binds the label is coated on magnetic beads and after the phenol/chloroform extraction of step (d) the product is incubated with the magnetic beads and the repetitive sequences of the probe are subtracted by magnetic force.

9. The method of claim 8, wherein the product is incubated with avidin-labeled magnetic beads in a binding buffer comprising about 1 M NaCl, PNM plus 2% BSA.

10. The method of claim 7, wherein after extraction with phenol and chloroform, a precipitate is formed in a mixture by addition of acetate and alcohol.

11. The method of claim 1, wherein the source nucleic acid molecule comprises amplified, microdissected chromosomal DNA.

12. The method of claim 1, wherein the source nucleic acid molecule comprises artificial chromosomes.

13. The method of claim 1, wherein the source nucleic acid molecule comprises a gene probe for detection of cancer.

14. The method of claim 13, wherein the cancer comprises leukemia, retinoblastoma, human Burkitt's lymphomas, ovarian cancer, uterine cancers, breast cancer, prostate cancer, or a combination thereof.

15. The method of claim 1, wherein the label is introduced by nick translation or PCR.

16. A repetitive sequences removed probe (RSRP) produced by the method of claim 1.

17. A nucleic acid molecule comprising a sequence represented by SEQ ID No: 2, SEQ ID No: 3, or a sequence substantially homologous to the SEQ ID No: 2, or SEQ ID No: 3.

18. A diagnostic test kit for detection of chromosomal abnormalities in a patient's sample comprising one or more repetitive sequences removed probes (RSRPs) that specifically detect chromosomal abnormalities and a detection agent comprising a detectable label.

19. The diagnostic test kit of claim 18, wherein the patient's sample comprises blood, saliva, plasma, serum, lymphoid fluid, or cerebrospinal fluid.

20. The diagnostic test kit of claim 18, wherein the repetitive sequences removed probes (RSRPs) are coupled to the detectable label.


SEQUENCE LISTING (11 sequences)

SEQ ID NO: 1 - DNA, 30 bp, Artificial Sequence
  degenerate primer
cgggagatccgactcgagnnnnnnatgtgg

SEQ ID NO: 2 - DNA, 32 bp, Artificial Sequence
  degenerate primer
ttcactgataccgactcgagnnnnnnatgtgg

SEQ ID NO: 3 - DNA, 20 bp, Artificial Sequence
  primer
ttcactgataccgactcgag

SEQ ID NO: 4 - DNA, 18 bp, Artificial Sequence
  primer
acaactaccaccgatggc

SEQ ID NO: 5 - DNA, 25 bp, Artificial Sequence
  primer
ttatttatcatattagttggattcg

SEQ ID NO: 6 - DNA, 17 bp, Artificial Sequence
  primer
tatggttggtattgctg

SEQ ID NO: 7 - DNA, 17 bp, Artificial Sequence
  primer
atggttccgccttatcg

SEQ ID NO: 8 - DNA, 19 bp, Artificial Sequence
  primer
ccagaccgtgacaatgatg

SEQ ID NO: 9 - DNA, 19 bp, Artificial Sequence
  primer
caagttctttcccctgcta

SEQ ID NO: 10 - DNA, 19 bp, Artificial Sequence
  primer
cgaaaaggttacaggacgg

SEQ ID NO: 11 - DNA, 19 bp, Artificial Sequence
  primer
caagttctttcccctgcta